(19) United States
(12) Patent Application Publication
(10) Pub. No.: US 2022/0330828 A1
(43) Pub. Date: Oct. 20, 2022

(54) SYSTEM FOR MEASURING THE PRESENCE AND/OR THE CONCENTRATION OF AN ANALYSIS SUBSTANCE IN A BODILY FLUID

(71) Applicant: TRUMPF Venture GmbH, Ditzingen (DE)

(72) Inventors: Andreas Popp, Markgroeningen (DE); Dieter Kraft, Gerlingen (DE)

(21) Appl. No.: 17/854,011

(22) Filed: Jun. 30, 2022

Related U.S. Application Data
(63) Continuation of application No. PCT/EP2020/087176, filed on Dec. 18, 2020.

(30) Foreign Application Priority Data
Dec. 30, 2019 (DE) 10 2019 135 877.9

(51) Int. Cl. A61B 5/00

(52) U.S. Cl. CPC A61B 5/0075; A61B 2562/06; A61B 2562/223

(57) ABSTRACT

A system for measuring the presence and/or the concentration of an analysis substance dissolved in a bodily fluid includes a light source configured to emit an excitation light, a detection device, and an optical device defining an excitation beam path of the excitation light from the light source to a measurement region of a sample and defining a detection beam path of a detection light from the measurement region of the sample to the detection device. The detection device is configured to detect the detection light and comprises a light-sensitive sensor and at least one filter element disposed in the detection beam path. The at least one filter element being configured to suppress a transmission of light with wavelengths outside of a specified analysis wavelength range about a selected IR absorption band that is specified as being characteristic for the analysis substance.

### CROSS REFERENCE TO RELATED APPLICATIONS

This application is a continuation of International Application No. PCT/EP2020/087176 (WO 2021/136699 A1), filed on Dec. 18, 2020, and claims benefit to German Patent Application No. DE 10 2019 135 877.9, filed on Dec. 30, 2019. The aforementioned applications are hereby incorporated by reference herein.

### FIELD

The invention relates to a system for measuring the presence and/or the concentration of an analysis substance, in particular blood sugar, dissolved in a bodily fluid, as per the preamble of claim 1.

### BACKGROUND

Such systems allow the presence and/or or the concentration of an analysis substance dissolved in bodily fluid, for example in intracellular or extracellular or interstitial fluid, to be determined—and to be precise preferably without a corresponding sample, for example a tissue or blood sample, having to be taken in advance (a so-called non-invasive measurement). By way of example, such a system allows the blood sugar level to be determined in the human blood. In particular, the measurement can be implemented in transdermal fashion, that is to say through the skin layers. In this way, the patient need not prick, e.g., a finger to take a drop of blood, which is regularly tedious and quite possibly uncomfortable.

The measurement of the presence and/or the concentration of the analysis substance using such a system is based on the analysis of the sample to be examined (for example, a tissue region below the human skin surface that contains bodily fluid) by means of infrared spectroscopy (IR spectroscopy). In this case, a measurement region of the sample is initially illuminated using excitation light in the infrared wavelength range, in particular between 1.5 μm and 25 μm. The light transmitted or reflected by the sample (detection light) is then spectrally analyzed. Characteristic molecular vibrations, in particular stretch vibrations and/or deformation vibrations of the atomic or molecular bonds, can be excited at certain wavelengths (energies) in the infrared wavelength range. The excitation light is absorbed to quite a significant extent by the analysis substance at these wavelengths. In this respect, the IR spectrum of the analysis substance has local absorption maxima (intensity minima of the detection light) at these wavelengths, which are referred to as “IR absorption bands”. Since an energy required to excite the vibrations and hence a wavelength of a respective IR absorption band is characteristic for a respective bond, a structure of the analysis substance can be deduced from analyzing the IR absorption bands. As it were, the IR absorption bands are a fingerprint of the analysis substance to be examined. In this respect, the presence of the analysis substance in the sample can be unambiguously determined from the presence or lack of presence of absorption bands at certain wavelengths. Moreover, a ratio of an intensity of the detection light at a wavelength of an IR absorption band to an intensity of the excitation light at this wavelength can supply information about a concentration of the analysis substance in the examined sample.

Spectrometer apparatuses, for example, are used in the prior art for the spectral analysis of the detection light. These usually comprise a diffractive or dispersive element for spatial-spectral splitting of the detection light and a spatially resolving detector for the wavelength-dependent detection of the split light. A disadvantage of systems of this type with spectrometers is that already relatively small changes in the relative position between the diffractive or dispersive element and the detector (e.g., as a result of a fall or temperature change) can lead to a displacement of the beam path and consequently to an incorrect measurement result. For this reason the system needs to be checked regularly and calibrated within the scope of servicing, which requires specialist knowledge. Moreover, a beam path of a certain length is required between dispersive/diffractive element and detector for a spectral split of the light, in order to be able to detect the spectral components of the detection light spatially separately from one another. Corresponding systems are therefore regularly comparatively large and can consequently have disadvantages during daily operation.

### SUMMARY

In an embodiment, the present disclosure provides a system for measuring the presence and/or the concentration of an analysis substance dissolved in a bodily fluid. The system includes a light source configured to emit an excitation light in an infrared wavelength range, a detection device, and an optical device configured to define an excitation beam path of the excitation light from the light source to a measurement region of a sample and configured to define a detection beam path of a detection light from the measurement region of the sample to a detection device. The detection device is configured to detect the detection light and comprises a light-sensitive sensor and at least one filter element arranged in the detection beam path, the at least one filter element being configured to suppress a transmission of light with wavelengths outside of a specified analysis wavelength range about a selected IR absorption band that is characteristic for the analysis substance.

### BRIEF DESCRIPTION OF THE DRAWINGS

Subject matter of the present disclosure will be described in even greater detail below based on the exemplary figures. All features described and/or illustrated herein can be used alone or combined in different combinations. The features and advantages of various embodiments will become apparent by reading the following detailed description with reference to the attached drawings, which illustrate the following:

FIG. 1 shows a sketched illustration of a system in a first configuration;

FIG. 2 shows a sketched illustration of a system in a second configuration;

FIG. 3 shows a sketched illustration of a system in a third configuration;

FIG. 4 shows a sketched illustration of a system in a fourth configuration;

FIG. 5 shows a detail denoted by V in FIGS. 3 and 4 an enlarged illustration; and

FIG. 6 shows a sketched illustration of a preferred configuration of a filter element in a plan view.

### DETAILED DESCRIPTION

The invention therefore considers the problem of facilitating, with little calibration outlay and a compact structure, a reliable analysis of an analysis substance dissolved in a bodily fluid. Moreover, a cost-effective embodiment is desirable.

Said object is achieved by a system having the features of claim 1.

The system overall is an apparatus, in the sense of an aggregate of things, of a plurality of devices which, in particular, are connected to form an apparatus or are integrated in a superordinate apparatus. The system serves to measure, especially in transdermal fashion, the presence and/or the concentration of an analysis substance dissolved in bodily fluid, in particular to determine a blood sugar concentration. Blood sugar is understood to mean, in particular, sugar, in particular glucose, dissolved in bodily fluid (for example in human blood).

The system comprises a light source for emitting excitation light, in particular broadband excitation light, in the infrared wavelength range. In particular, the light source is designed to emit excitation light at wavelengths above 1.5 μm, in particular between 1.5 μm and 25 μm.

The system comprises an optical device which for the excitation light defines an excitation beam path from the light source to a measurement region of a sample and which for detection light from the measurement region of the sample defines a detection beam path from the measurement region of the sample to a detection device. The optical unit can comprise a plurality of optical elements (e.g., lenses, reflectors, prisms, stops, optical fibers), by means of which the excitation beam path and the detection beam path are defined. In particular, the sample can be a tissue region which contains a bodily fluid and is located below the human skin surface, for example in the region of an arm or a finger.

The system further comprises a detection device for detecting the detection light. The detection device comprises a light-sensitive sensor which in particular is designed to produce measurement signals, preferably electrical measurement signals, from the captured light.

The detection device moreover comprises at least one filter element arranged in the detection beam path, the at least one filter element being designed to suppress a transmission of light with wavelengths outside of a predefined analysis wavelength range about an IR absorption band which is specified as being characteristic for the analysis substance. In this respect, the at least one filter element is designed in such a way that the wavelengths of the detection light not relevant to an analysis of the respective IR absorption band can be substantially filtered out, i.e., not be transmitted to the sensor. In particular, the at least one filter element is designed to suppress a transmission of light at wavelengths outside of the specified analysis wavelength range about a respective selected absorption test wavelength of the absorption light, that which absorption test wavelength it is possible to excite a stretch and/or deformation vibration of an atomic or molecular bond of the analysis substance that is characteristic for the chemical structure of the analysis substance.

Such a configuration already facilitates an approximate evaluation to the effect of whether the analysis substance is present in the sample with a certain threshold concentration. The reliability of the measurement can be further increased if there is a further spectral resolution of the respective IR absorption band, as still explained in more detail below. Moreover, a system of this type has a compact structure since beam paths which are required, for example in spectrometer apparatuses, for the spatial split of a beam into wavelength components can be dispensed with. Blood sugar measuring appliances having a system of this type can have a comparatively small embodiment, simplifying the handling thereof. Moreover, blood sugar measuring appliances having a system of this type are particularly robust. In particular, there is reduced outlay for maintenance since regular calibration of a spectrometer—as usually required in the case of the systems known from the prior art—is dispensed with. This is particularly advantageous since diabetics must regularly—and consequently also on the go or when traveling—monitor the blood sugar level. The system is also comparatively cost-effective since there is no need for elements with a spectrally splitting effect such as, e.g., gratings, which are comparatively expensive on account of the high precision required.

It is possible that only one characteristic IR absorption band of the analysis substance should be analyzed. Then, the at least one filter element can be designed to suppress a transmission of light with wavelengths outside of an analysis wavelength range about this IR absorption band. It is also possible that a plurality of IR absorption bands should be analyzed. Then, the at least one filter element can be designed to suppress a transmission of light with wavelengths outside of a respective analysis wavelength range about a respective IR absorption band to be analyzed.

It is conceivable for light source and detection device to be arranged on opposite sides of the sample (transmission configuration). Then the detection light in particular comprises at least a portion of the excitation light transmitted by the sample. It is also conceivable for light source and detection device to be arranged on the same side of the sample (reflection configuration). Then the detection light in particular comprises at least a portion of the excitation light reflected by the sample.

Preferably, the respective analysis wavelength range is above 1.5 μm, in particular between 1.5 μm and 25 μm, further particularly between 1.5 μm and 3 μm. The IR absorption bands of glucose dissolved in human blood which are relevant to the determination of the concentration are located in this wavelength range. It is particularly preferable for the analysis wavelengths to be a sub-range of the wavelength range from 1.7 μm to 2.6 μm.

Further, it is preferable for the respective analysis wavelength range to be within a wavelength range of ±50 nm, more particularly ±10 nm, further particularly ±5 nm, further particularly ±2.5 nm, further particularly ±1 nm, about the selected, characteristic IR absorption band of the analysis substance, more particularly about a specified IR absorption band of glucose as a solution in human bodily fluid. This facilitates a selective analysis of the specified IR absorption band of the analysis substance while avoiding further disturbance signals, for example as a result of further IR absorption bands. By way of example, the relevant IR absorption bands of glucose are at 2140 nm, 2270 nm and 2330 nm.

Preferably, the at least one filter element is arranged and/or designed in such a way that a component of the light in the detection beam path strikes the sensor, in particular a portion of the sensor, as reference light which remained uninfluenced by the at least one filter element. In particular, the at least one filter element is arranged and/or designed in such a way that at least one component of the excitation light that was elastically scattered by the sample strikes the sensor. This allows the light source to be checked in respect of functionality and/or allows the calibration of the optical device to be checked. For this purpose it is possible that a portion of the sensor is not covered by the at least one filter element. Then, light can strike the sensor past the at least one filter element.

In an advantageous configuration, the at least one filter element is designed as a component with a planar extent. Then there is no need for precise spatial focusing of the detection light, which may further reduce calibration outlay. In particular, the at least one filter element extends in a plane. By way of example, it is possible for the at least one filter element to have a slab-like embodiment.

In particular, the sensor comprises a detecting sensor surface. Particularly preferably the at least one filter element is arranged on the sensor surface. In particular, the sensor surface can have a planar embodiment; then the at least one filter element can be arranged on the plane sensor surface in slab-like fashion. This facilitates a planar detection of the detection light, and so there is no need to focus the detection light on a certain region of the sensor surface. Further, a particularly compact structure of the detection device is obtained by virtue of the fact that the at least one filter element is arranged on the sensor surface.

It is particularly preferred for the at least one filter element and the sensor to be securely connected to one another, in particular to be embodied in monolithic form or to be joined into a pre-assembled component part. In this respect, there cannot be an inadvertent shift or disturbance of the beam path between filter element and sensor. In this way, a particularly robust structure is obtained, as a result of which it is possible to eliminate the risk of an unwanted maladjustment—for example as a result of the system falling on the ground—and hence it is possible to minimize calibration outlay. By way of example, it is possible for corresponding filter structures to be vapor deposited on the sensor surface or to be produced on the latter by lithography.

In a particularly preferred configuration, the sensor comprises an array of light-sensitive pixels which are designed to detect incident light. By way of example, it is possible for the light-sensitive pixels to be arranged in rows and columns. Further, it is preferable for the at least one filter element to have a plurality of narrowband filter regions. Preferably, the narrowband filter regions are designed to suppress a transmission of light with wavelengths outside a pass range about a center wavelength. In particular, the narrowband regions act as bandpass filters for a corresponding wavelength range which is narrow in comparison with the analysis wavelength range.

Further, it is preferable for one filter region to be respectively assigned to one pixel group of adjacently arranged pixels, in particular to one pixel. Preferably, a filter region is arranged on each pixel. In particular, the filter regions and their assigned pixel groups or pixels are arranged relative to one another in such a way that along the detection beam path light passing through a respective filter region is only captured by the respectively assigned pixel group or by the respectively assigned pixel. By way of example, it is possible for the filter regions of the at least one filter element to be arranged in rows and columns, with the filter regions being arranged in such a way that a filter region is arranged in front of each pixel of the sensor.

In particular, it may also be advantageous if no filter region is arranged in front of at least one pixel or at least one pixel group. Then, a component of the light in the detection beam path, in particular the excitation light which was elastically scattered by the sample, can strike this at least one pixel and, as explained above, be detected as reference light. By way of example, it is conceivable for the at least one filter element to have a local cutout corresponding to the size of a pixel or the size of a pixel group. It is also conceivable for no filter structure to have been vapor deposited on the at least one pixel, for example in an edge region of the sensor or the sensor surface.

It is possible for all filter regions to have the same center wavelength. However, it is preferable for filter regions to be provided with center wavelengths that differ from one another. In particular, the center wavelengths can be chosen such that different spectral ranges are detected using only one sensor. This renders it possible to approximate a spectral curve of the IR absorption band to be analyzed and thus to distinguish whether for example a measured intensity minimum (absorption maximum) represents an IR absorption band or merely a disturbance signal. Such a configuration also allows a plurality of characteristic IR absorption bands to be analyzed using only one sensor and one filter element. Here, this can relate to a plurality of IR absorption bands of the analysis substance—which may be advantageous for example for a precise determination of the concentration of the analysis substance in the bodily fluid—and/or for characteristic IR absorption bands of different substances (e.g., glucose and lactate or medical active ingredients) dissolved in the bodily fluid.

It is possible that each filter region of the filter element has a different center wavelength. Then, a filter region with a different center wavelength can be assigned to each pixel/each pixel group of the sensor. It is particularly preferred for a plurality of filter regions of the filter element to have the same center wavelength, in particular the same pass ranges. Then, filter regions with the same center wavelengths, in particular the same pass ranges, can be assigned to a plurality of pixels/pixel groups. In this way, light of a certain wavelength can be detected by a plurality of pixels, which has a positive effect on the signal-to-noise ratio.

To facilitate a detailed analysis of the IR absorption band to be examined (e.g., the shape, full width at half maximum, wavelength at the absorption maximum etc., thereof), it is preferable for the center wavelengths to be distributed at spectrally spaced apart intervals over the (respective) analysis wavelength range. Preferably, the intervals are distributed equidistantly. It is particularly preferable for the intervals between the center wavelengths of the filter regions assigned to a respective analysis wavelength range to be less than 5 nm, preferably less than 2 nm, further preferably less than 1 nm, further preferably less than 0.5 nm, further preferably less than 0.2 nm. Given a specified number of pixels, a smaller measurement interval is advantageous for a higher spectral resolution (more measurement points in the analysis wavelength range). By contrast, a larger interval is advantageous for a higher signal-to-noise ratio (more pixels detect light of the same wavelength).

Preferably, the filter regions assigned to a respective analysis wavelength range from a filter group which repeats, preferably repeats periodically, over the filter element. It is possible that only one characteristic IR absorption band of the analysis substance should be analyzed. In this case, a single filter group can be provided, which is formed by the filter regions assigned to the analysis wave range of this IR absorption band. This filter group can then repeat periodically, for example like a line or mosaic, over the filter element. It is also possible that a plurality of characteristic IR absorption bands should be analyzed (for example a plurality of IR absorption bands of the analysis substance or IR absorption bands of different substances dissolved in bodily fluid). In this case, the filter element can have a plurality of different filter groups, with the respective filter group being formed by the filter regions assigned to a respective analysis wavelength range—that is to say one filter group is respectively assigned to one respective analysis wavelength range.

It is possible for the filter regions of the filter element to be arranged in rows and columns. Then, the filter regions of the filter group can be arranged along a row/column, which repeats along the rows/columns (line pattern). However, the filter regions of a filter group can extend over the same number of columns and rows, preferably over two columns and two rows, further preferably over four columns and four rows, in particular over five columns and five rows. This filter group can then repeat like a mosaic over the filter element (mosaic pattern).

The sensor of the detection device is preferably a semiconductor sensor, for example based on GaSb, InGaAs, PbS, PbSe, InAs, InSb or HgCdTe. Such sensors are distinguished in particular by a high sensitivity to light with wavelengths in the infrared range which is relevant to the analysis of IR absorption bands, in particular from blood sugar. Moreover, such sensors are available in comparatively cost-effective fashion, which is advantageous for the use of the system according to the invention having such a sensor as a mass-market product. By way of example, the sensor can be in the form of a photodiode.

For a detailed spectral analysis of an IR absorption band, it is further advantageous for the excitation light to have a certain spectral width, that is to say comprise light components of different wavelengths. In particular, the light source is designed in such a way that a spectral width of the excitation light emitted thereby (that is to say a wavelength interval of the electromagnetic spectrum in which the excitation light has a non-vanishing intensity) has a size greater than 10 nm, in particular greater than 50 nm, further particularly greater than 100 nm, further particularly greater than 500 nm, further particularly greater than 1 μm. Such a configuration moreover renders it possible to analyze IR absorption bands at different wavelengths (e.g., a plurality of IR absorption bands of the analysis substance or IR absorption bands of different substances) using only one light source.

Preferably the light source is a laser light source. Laser light is distinguished by a high luminous intensity, which is advantageous for analyzing analysis substances which are present only in comparatively low concentrations.

The laser light source may be in the form of a laser diode array which comprises a plurality of laser diodes for emitting laser light. The laser diodes can preferably be semiconductor diodes, for example based on GaSb or InP. In particular, the laser diode array is designed in such a way that the laser beams emitted by the individual laser diodes are superposed to form the excitation light. By way of example, it is conceivable that the laser diodes are arranged next to one another on a base plate. It is also conceivable for the laser light source to be in the form of a monolithic laser diode array. To produce excitation light with a given spectral width, it is preferable for the laser diodes of the laser diode array to be designed to output laser light with a different central wavelength in each case. Preferably, the laser diodes are designed in such a way that the emission spectra of the laser diodes spectrally overlap in certain regions. Then, a spectral intensity distribution of the excitation light is particularly homogeneous.

It is also possible that the laser light source comprises at least one quantum cascade laser which is designed to simultaneously emit laser light with different central wavelengths. In this respect, the at least one quantum cascade laser is especially designed such that electron transitions may occur, within the scope of which photons with different energies (wavelengths) are emitted in each case. Preferably, the at least one quantum cascade laser is designed such that there are a multiplicity of electron transitions between energy levels with the same energy difference. Then, an intensity of the emitted laser light is particularly high.

It is also possible that the laser light source comprises at least one interband cascade laser.

It is also possible that the laser light source comprises at least one multi-quantum well diode which has a plurality of multi-quantum well regions for the emission of laser light. Preferably, the individual multi-quantum well regions are arranged in a single laser chip. Multi-quantum well diodes are distinguished by a comparatively high efficiency at a low threshold current. By way of example, GaSb, GaAs or InP are conceivable as a base material for such multi-quantum well diodes. Preferably, the multi-quantum well regions are designed such that they each emit laser light with a different central wavelength. Moreover, it is preferable for the emission spectra of the multi-quantum well regions to spectrally overlap in certain regions. Then, a spectral intensity distribution of the excitation light is particularly homogeneous.

Preferably, the system further comprises a control device. In particular, the control device comprises a non-volatile memory, in which one or more reference spectra are savable or saved. In this case, the at least one reference spectrum preferably comprises at least the wavelength of the respective selected IR absorption band of the analysis substance and/or the wavelengths of the specified analysis wavelength range about the respective selected IR absorption band. It is possible for a reference spectrum to be the emission spectrum of the light source, in particular the spectrum of the excitation light. It is also possible for a reference spectrum to be an IR spectrum of the bodily fluid to be examined or of a reference solution that is similar to this bodily fluid. Further, it is conceivable for a reference spectrum to be an IR spectrum of the analysis substance as a solution with a certain concentration in the bodily fluid to be examined or in a reference solution similar to this bodily fluid, for example an IR spectrum of glucose as a solution in human blood. Inter alia, this allows a measured intensity of the detection light to be normalized and thus allows a concentration of the analysis substance to be determined (absolutely) in the examined sample.

Within the scope of an advantageous configuration of the system, the optical device may comprise at least one first optical fiber or waveguide which is designed to guide the excitation light on at least a portion of its optical path from the light source to the measurement region of the sample. In this case and in the present context, waveguide more particularly denotes a silicon oxide or silicon nitride applied in defined fashion to a silicon substrate, as is typically implemented in semiconductor technology. Moreover, the optical device can comprise at least one second optical fiber or waveguide which is designed to guide the detection light on at least a portion of its optical path from the measurement of the sample to the detection device. Such a configuration facilitates a precise definition of the beam path for the excitation light or the detection light, in particular the guidance of the excitation light or the detection light along curves even without additional optical means such as mirrors, for example, promoting a compact design of the system. Moreover, the risk of an unwanted maladjustment of the beam path can be reduced and a calibration outlay can consequently be minimized. In one configuration with optical fibers, the optical device may then optionally comprise input coupling and/or output coupling means for input coupling and/or output coupling of light into the respective optical fiber, for example in the form of appropriately designed lens means.

To be able to guide the light in the infrared wavelength range as a loss-free as possible, it is preferable for the at least one first optical fiber and/or the at least one second optical fiber to be in the form of a hollow fiber. In this respect, the at least one first optical fiber and/or the at least one second optical fiber are preferably designed as fibers, preferably cylindrical fibers, in particular, which in the cross section have at least one continuous cavity along their longitudinal extent. By way of example, such a hollow fiber can be manufactured from a polymer or from a glass, more particularly from fused quartz (fused silica).

In the following description and the drawing, the same reference signs are used in each case for identical or corresponding features.

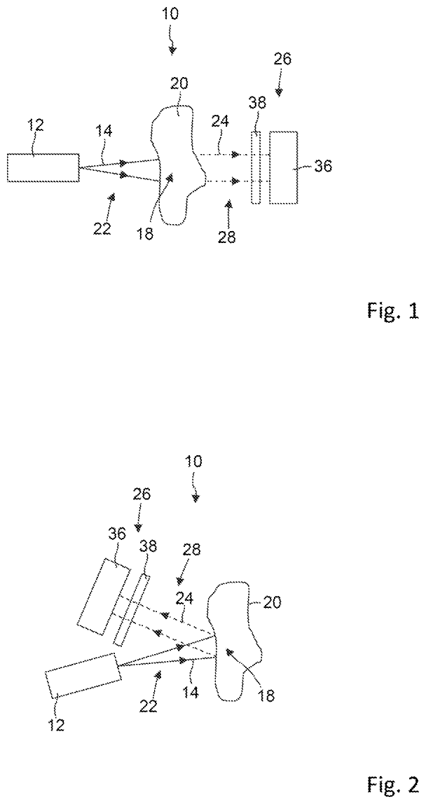

FIGS. 1 to 4 show sketched illustrations of different configurations of a system 10 for measuring the presence and/or the concentration of an analysis substance dissolved in a bodily fluid. In particular, the system 10 is designed to determine a concentration of sugar, in particular glucose, dissolved in a bodily fluid.

The system 10 comprises a light source 12 for emitting excitation light 14 in the infrared wavelength range, in particular with wavelengths between 1.5 μm and 25 μm. By way of example, the light source 12 can be a laser diode array having a plurality of semiconductor laser diodes, which each emit laser light with a different central wavelength. It is also possible that the light source 12 comprises at least one quantum cascade laser which is designed to simultaneously emit laser light in the infrared wavelength range. Further, it is conceivable that the light source 12 comprises a multi-quantum weld diode.

The system 10 moreover comprises an optical device 16 which is designed to guide the excitation light 14 from the light source 12 to a measurement region 18 of a sample 20, for example a blood-containing tissue region in a human body. To this end, the optical device 16 may in particular comprise one or more optical elements 30 for beam deflection and/or beam guidance, which for the excitation light 14 define an excitation beam path 22 from the light source 12 to the measurement region 18 of the sample 20 (cf. FIG. 4).

The optical device 16 is further designed to guide detection light 24 from the measurement region 18 of the sample 20 to a detection device 26. To this end, the optical device 16 may in particular comprise one or more optical elements 32, 34 for beam deflection and/or beam guidance, which for the detection light 24 define a detection beam path 28 from the measurement region 18 of the sample 20 to the detection device 26 (cf. FIG. 4).

It is possible for the excitation light 14 and/or the detection light 24 to propagate as a free beam (cf. FIGS. 1 to 3). Then, the optical device 16 can in particular comprise optical elements in the form of lenses, reflectors, deflection mirrors, prisms or the like (not depicted here) in order to define the excitation beam path 22 and the detection beam path 28

It is also possible for the excitation light 14 and/or the detection light 24 to be guided along their respective optical path by means of optical fibers 30, 32 (cf. FIG. 4). Then, the optical device 16 may for example comprise a first optical fiber 30 which for the excitation light 14 defines the excitation beam path 24 for at least a portion of its optical path 14 from the light source 12 to the measurement region 18 of the sample 20 (shown in a sketched representation in FIG. 4). Moreover, the optical device 16 can comprise a second optical fiber 32 which for the detection light 24 defines the detection beam path 28 for at least a portion of its optical path from the measurement region 18 of the sample 20 to the detection device 26. As depicted in exemplary fashion in FIG. 4 for the second optical fiber 32, the optical device 16 may then moreover comprise one or more input/output coupling means 34, for example in the form of lens means, for input/output coupling the excitation light 14 or the detection light 24 into the respective optical fiber 30, 32. In exemplary and preferred fashion, the optical fibers 30, 32 are in the form of hollow fibers.

FIG. 1 shows the system 10 in a transmission configuration, in which the light source 12 and the detection device 26 are arranged on opposite sides of the sample 20. In such configuration, the detection light 24 comprises at least a portion of the excitation light 14 transmitted by the sample 20.

FIGS. 2 to 4 show the system 10 in a reflection configuration, in which the light source 12 and the detection device 26 are arranged on the same side of the sample 20. In such a configuration, the detection light 24 then comprises at least a portion of the excitation light 14 reflected by the sample 20.

To detect the detection light 24, the detection device 26 comprises a light-sensitive sensor 36 which is designed to produce electrical measurement signals from the captured light. In exemplary and preferred fashion, the sensor 36 is a semiconductor sensor which is designed to detect light with wavelengths in the infrared range.

The detection device 26 further comprises a filter element 38, which is arranged in the detection beam path 28 between the sample 20 and the sensor 36. In exemplary and preferably fashion, the filter element 38 is designed overall as a component with a planar extent, and it extends substantially within a plane. The filter element 38 is embodied to suppress (see above) a transmission of light with wavelengths outside of a specified analysis wavelength range about a respective specified IR absorption band of a selected analysis substance, in particular blood sugar (glucose) dissolved in human blood.

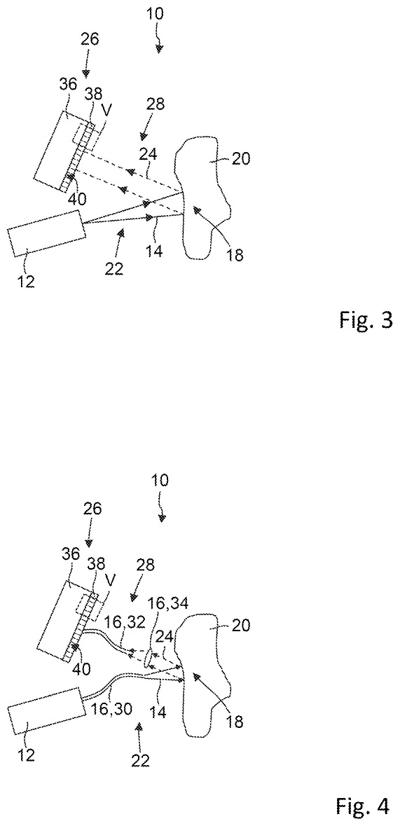

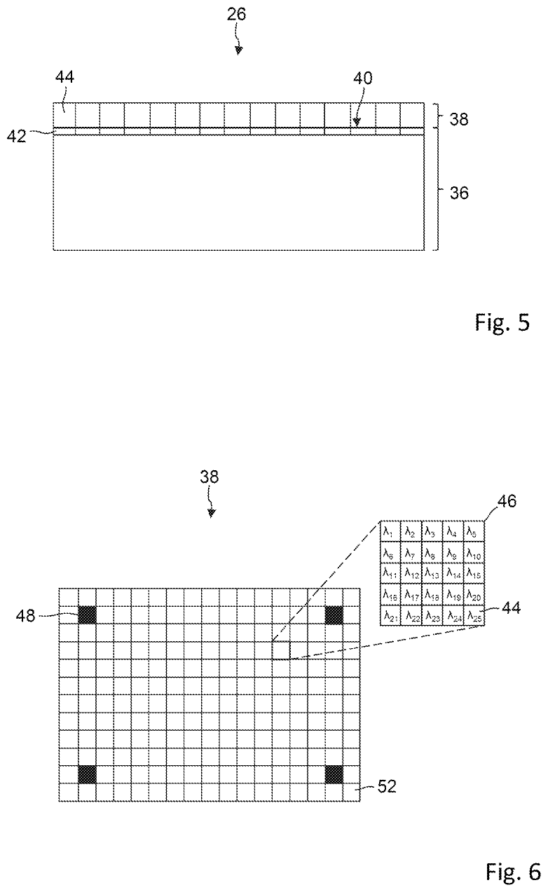

In the case of a preferred configuration of the detection device 26, as depicted in FIGS. 3 and 4, the sensor 36 comprises a detecting sensor surface 40, which likewise has a planar form. The sensor surface 40 comprises an array of light-sensitive pixels 42 which are arranged in rows and columns in a manner known per se and are therefore not explained in more detail (cf. FIG. 5). Then, the filter element 38 is preferably arranged on the sensor surface 40 of the sensor 36. In particular, the filter element 38 is connected, in particular integrally connected, to the sensor 36 to form a permanently assembled component part.

FIG. 6 shows a preferred configuration of the filter element 38 in a top view. The filter element 38 has a plurality of narrowband filter regions 44, which are arranged in rows and columns. In this case, the filter regions 44 are arranged in such a way that a filter region 44 is arranged in front of each pixel 42 of the sensor 36 (cf. FIG. 5). In exemplary and preferred fashion, the pixels 42 and the filter regions 44 have the same dimensions as viewed orthogonally on the sensor surface 40. In this respect, a respective filter region 44 covers the entire detection surface of the pixel 42 assigned thereto, in particular it covers exclusively this pixel and no other pixel. Preferably, the filter regions 44 are formed in monolithic fashion with the respective pixels 42. In exemplary and preferred fashion, the filter regions 44 are formed by filter structures which are vapor deposited on the sensor surface 40 of a respective pixel 42, or which have been produced thereon by lithography.

In the exemplary configuration of the filter element 38 illustrated in FIG. 6, 25 (5×5) filter regions 44 together form a filter group 46, with the filter group 46 repeating over the filter element 38 in mosaic-like fashion. The filter regions 44 of the filter group 46 have center wavelengths λ₁ to λ₂₅ which deviate from one another and which are in an analysis wavelength range about the IR absorption band of the analysis substance to be analyzed. In this respect, 25 different spectral ranges (bands) in the analysis wavelength range can be detected independently of one another in the shown configuration.

The center wavelengths λ₁ to λ₂₅ are preferably distributed in equidistant intervals over the analysis wavelength range. By way of example, it is conceivable for the analysis wavelength range to be a range of ±2.5 nm about the specified IR absorption band. In this case, an interval of 0.2 nm arises when there is a total of 25 filter regions 44.

In configurations not illustrated here, it is also possible for the filter regions 44 assigned to a respective analysis wavelength range to repeat over the filter element 38 in irregular fashion.

Optionally, no filter region 46 may be arranged in front of one or more pixels 42 of the sensor 36. Then, the excitation light 14 reflected or transmitted by the sample 20 can be detected as reference light by these pixels 42. By way of example, it is conceivable for a local cutout that corresponds to the size of a pixel or a pixel group to be provided in the filter element 36 (this is depicted schematically in FIG. 6 by regions 48 shaded in black). These regions 48 are preferably arranged in an edge region of the filter element 38.

To analyze a plurality of IR absorption bands, the filter element 38 may comprise a plurality of different filter groups 46, which are each assigned one of the IR absorption bands to be analyzed. Then, the filter groups 46 are in particular designed in such a way that the filter regions 44, which form a respective filter group 46, are within the analysis wavelength range that includes a respective IR absorption band. The different filter groups 46 can then repeat over the filter element 38, for example in an alternating mosaic-like fashion.

While subject matter of the present disclosure has been illustrated and described in detail in the drawings and foregoing description, such illustration and description are to be considered illustrative or exemplary and not restrictive. Any statement made herein characterizing the invention is also to be considered illustrative or exemplary and not restrictive as the invention is defined by the claims. It will be understood that changes and modifications may be made, by those of ordinary skill in the art, within the scope of the following claims, which may include any combination of features from different embodiments described above.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

1. A system for measuring the presence and/or the concentration of an analysis substance dissolved in a bodily fluid, the system comprising:
a light source configured to emit an excitation light in an infrared wavelength range;
a detection device; and
an optical device configured to define an excitation beam path of the excitation light from the light source to a measurement region of a sample and configured to define a detection beam path of a detection light from the measurement region of the sample to a detection device;
wherein the detection device is configured to detect the detection light and comprises a light-sensitive sensor and at least one filter element arranged in the detection beam path, the at least one filter element being configured to suppress a transmission of light with wavelengths outside of a specified analysis wavelength range about a selected IR absorption band that is characteristic for the analysis substance.

2. The system as claimed in claim 1, wherein the analysis wavelength range is above 1.5 μm.

3. The system as claimed in claim 1, wherein the analysis wavelength range is within a wavelength range of ±50 nm about the selected IR absorption band of the analysis sub stance.

4. The system as claimed in claim 1, wherein the at least one filter element is configured in such a way that a component of the detection light in the detection beam path strikes the sensor, as reference light uninfluenced by the at least one filter element.

5. The system as claimed in claim 1, wherein the at least one filter element has a planar extent extending in one plane.

6. The system as claimed in claim 1, wherein the sensor comprises a detecting sensor surface and wherein the at least one filter element is disposed on the sensor surface.

7. The system as claimed in claim 1, wherein the sensor comprises an array of light-sensitive pixels and wherein the at least one filter element has a plurality of narrowband filter regions, wherein each filter region is respectively assigned to one pixel group of adjacently arranged pixels in such a way that along the detection beam path light passing through a respective filter region is only captured by the respectively assigned pixel group.

8. The system as claimed in claim 7, wherein the filter regions have center wavelengths that differ from one another are provided and wherein the center wavelengths are distributed at equidistant intervals over the respective analysis wavelength range.

9. The system as claimed in claim 8, wherein the filter regions assigned to an analysis wavelength range from a filter group which repeats over the filter element.

10. The system as claimed in claim 1, wherein the light source is configured such that a spectral width of the excitation light is greater than 10 nm.

11. The system as claimed in claim 1, wherein the light source is in the form of a laser diode array comprising a plurality of laser diodes, each with a central wavelength and an emission spectra, wherein the central wavelength of each laser diode differs from the others and wherein the emission spectra of each laser diode overlaps with the emission spectra of at least one other of the laser diodes.

12. The system as claimed in claim 1, wherein the light source comprises at least one quantum cascade laser configured to simultaneously emit a laser light having different central wavelengths.

13. The system as claimed in claim 1, where in the light source comprises at least one multi-quantum well diode comprising a plurality of multi-quantum well regions each emitting a laser light with a different central wavelength.

14. The system as claimed in claim 1, further comprising a control device having a non-volatile memory containing one or more reference spectra comprising the wavelength of the selected IR absorption band of the analysis substance.

15. The system as claimed in claim 1, wherein the optical device comprises at least one first optical fiber configured to guide the excitation light on at least a portion of its optical path from the light source to the measurement region of the sample,
and/or at least one second optical fiber, configured to guide the detection light on at least a portion of its optical path from the measurement of the sample to the detection device.
